(19) United States
(12) Patent Application Publication
(10) Pub. No.: US 2019/0183401 A1
(43) Pub. Date: Jun. 20, 2019

(54) Hemoencephalography Neurofeedback Device

(71) Applicant: John Witzigreuter, Canton, GA (US)

(72) Inventor: John Witzigreuter, Canton, GA (US)

(21) Appl. No.: 16/223,380

(22) Filed: Dec. 18, 2018

Related U.S. Application Data
(60) Provisional application No. 62/607,473, filed on Dec. 19, 2017.

(51) Int. Cl. A61B 5/00; A61B 5/026; A61M 21/02

(52) U.S. Cl. CPC A61B 5/4064; A61B 5/0261; A61B 5/486; A61B 5/6803; A61M 2021/005; A61B 2562/0238; A61B 2562/043; A61B 2562/164; A61M 21/02

(57) ABSTRACT

A hemoencepholagraphy neurofeedback device includes a headband assembly, and a circuit board coupled to said headband strap. The circuit board includes at least a pair of light emitting diodes, a receiver and a microprocessor. The pair of diodes includes a red light and an infrared light. When the pair of light emitting diodes are emitted onto a user's forehead, the receiver measures the amount of light returned from the forehead, and a display device or software application displays the microprocessor's calculation of the amount of blood flow in the area of the user's forehead. The neurofeedback device may be wirelessly connected to a mobile device or software application. The neurofeedback device allows the user to quantify the amount of blood flow in the brain, causing the growth of new blood vessels in the brain in the areas where the neurofeedback is used.

### CROSS-REFERENCE TO RELATED APPLICATIONS

The present application claims priority under 35 USC § 119 on pending U.S. Provisional Application Ser. No. 62/607,473 filed on Dec. 19, 2017, the disclosure of which is incorporated by reference.

### BACKGROUND

Hemoencepholagraphy (“HEG”) biofeedback (also known as HEG neurofeedback) was developed by Hershel Toomim and is best described in his U.S. Pat. No. 5,995,857. HEG feedback involves measuring neural activity based on neurovascular coupling. Neurovascular coupling is the mechanism by which cerebral blood flow is matched to metabolic activity. For example, when a region of the cortex is used in a specific cognitive task, neuronal activity in that region increases, consequently increasing local metabolic rate. To keep up with the nutritional and waste removal demands of a higher metabolic rate, cerebral blood flow to the cortical area in use is thought to need to increase proportionally. Along with the increase in flow, hemoglobin molecules in the blood, which are responsible for the transport and transference of oxygen to tissue throughout the body, must increase the amount of oxygen they deliver to the activated region of the cortex, resulting in a greater local blood oxygenation level. This is also referred to as the haemodynamic response.

Currently there are two forms of HEG neurofeedback. The first is accomplished via near-infrared (also referred to as nIR), which utilizes red and infrared light to determine blood flow. The second is passive infrared (also referred to as pIR) which is passive infrared HEG and measures the heat given off by the brain due to metabolic activity.

Near-infrared HEG neurofeedback works by shining red and infrared light into a subject's prefrontal cortex to determine the amount of blood flow in that region of the brain. The measurements taken are then converted into a value that may be displayed on a screen in both graphical and numeric form.

When the subject sees the numeric representation he or she can make the number increase by simply thinking about it and staying in a relaxed concentration state.

These existing technologies are connected to a computer and typically require an administrator to assist the subject in his/her training or measuring sessions. These solutions are typically cumbersome, uncomfortable and can be hard for the subject to use and interpret.

The present disclosure is, amongst other things, directed to providing a solution that solves these prior art drawbacks and to providing a cost effective, comfortable and easy to use solution for obtaining, reporting, and interpreting HEG biofeedback.

### BRIEF DESCRIPTION OF THE DISCLOSURE

The present disclosure will become more fully apparent from the following description and drawing figures. Understanding that the accompanying drawings depict only exemplary embodiments of the disclosure, and are, therefore, not to be considered to be limiting of the scope of the present disclosure, the drawings will be described and explained with specificity and detail in reference to the accompanying description as exemplary embodiments.

In an embodiment, the present disclosure comprises a nIR HEG neurofeedback device that has a wireless connection to an Android or iOS application (or “app”) via low energy Bluetooth (BLE). The device may easily connect to the app, allowing a user to do an HEG training session at home or in any distraction-free environment.

In an embodiment, the HEG device consists of a cushioned strap that is adjustable. Embedded into the cushioned strap may be a circuit board with a battery or other power source to power the device.

In an embodiment of an operational phase of the device, the operation may commence with the user opening the app and placing the headband on his or her forehead in an fp1, fp2 or fpz location. The device may be configured to automatically sync with and operationally connect to the app. The user may thereafter specify the duration of a session, and then initiate a training session or sessions.

A goal of this disclosure is to provide for an HEG neurofeedback device that can easily be used at home, is effective and quantifiable. This is accomplished by simplifying the operation of the device as much as possible and incorporating controls, data collection, etc. into an easy-to-use computer or mobile device application.

### DESCRIPTION OF THE DRAWINGS

The advantages and features of the present disclosure will become better understood with reference to the following detailed description and claims taken in conjunction with the accompanying drawings, wherein like elements are identified with like symbols, and in which:

FIG. 1 shows an exploded view of an HEG headband assembly, in accordance with an exemplary embodiment of the present disclosure;

FIG. 2 shows another exploded view of an HEG assembly, in accordance with an exemplary embodiment of the present disclosure:

FIG. 3 shows a circuit board of an HEG headband assembly, in accordance with an exemplary embodiment of the present disclosure;

FIGS. 4, 5, 6 and 7 show exemplary screenshots of a software application for a HEG assembly, in accordance with an exemplary embodiment of the present disclosure;

FIG. 8 shows exemplary training or usage for an HEG assembly, in accordance with an exemplary embodiment of the present disclosure;

FIG. 9 shows an exemplary training schedule for using an HEG assembly in accordance with an exemplary embodiment of the present disclosure; and

FIG. 10 shows an exemplary alternate configuration for an HEG headband, in accordance with an exemplary embodiment of the present disclosure.

Like reference numerals refer to like parts throughout the description of several views of the drawings, in which numerals refer to:
1) HEG Headband Assembly
2) Wireless Application
3) Headband Strap
4) Loop Fabric
5) Hook Fabric
6) Front Plate
7) Headband Foam
8) Circuit Board
9) T5 Plastic Snap Back
10) T5 Plastic Male Snap
11) T5 Plastic Female Snap
12) Battery
13) Battery Holder
14) Serpentine Flex Joint
15) LED Pair
16) Receiver
17) PCB Board

### DETAILED DESCRIPTION OF THE DISCLOSURE

The exemplary embodiments described here in detail are for illustrative purposes and are subject to many variations in structure and design. It will be apparent, however, that the present disclosure is not limited to a device as shown and described. It is understood that various omissions and substitutions of equivalents are contemplated as circumstances may suggest or render expedient, but these are intended to cover the application or implementation without departing from the spirit or scope of the claims of the present disclosure.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

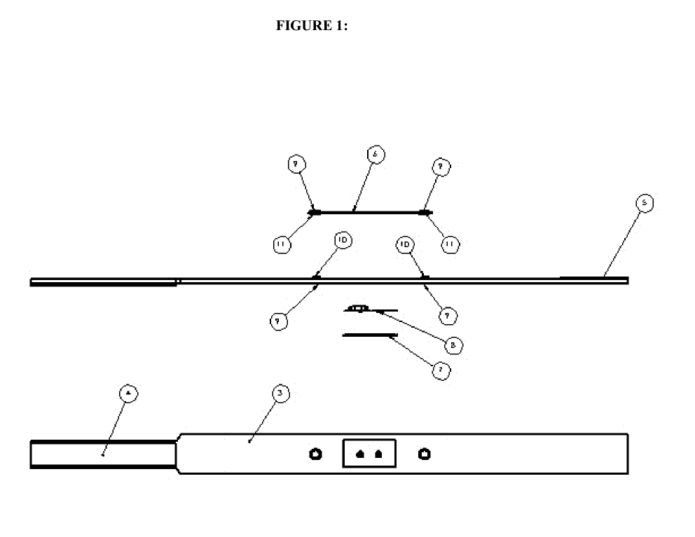

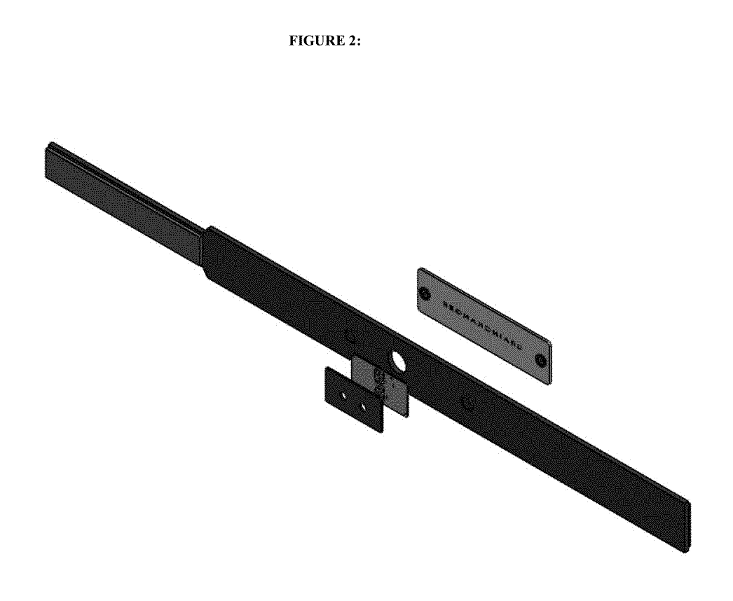

FIGS. 1 and 2 show exploded views of an HEG headband assembly 1 in accordance with an exemplary of the present disclosure. In an embodiment the assembly comprises a headband strap 3, which strap is configured to allow for adjustment in size of the headband. In an embodiment, the strap may be comprised of a hook and loop fabric (such as Velcro) for size adjustments. The headband preferably further comprises a circuit board 8.

The headband strap 3 can be made from a variety of materials including plastic, rubber, fabric, foam or the like. The preferred material is a fabric bonded neoprene foam that is flexible, padded, and has absorbent qualities. Loop fabric 4 is sewn or glued to or near a first side or end of the headband strap 3 and hook fabric 5 is sewn to or near a second side or end of the strap. The hook and loop fabrics provide a convenient way to adjust the headband to fit on a variety of head sizes. It will be apparent that the headband strap 3 must be large enough to circumvent a variety of head sizes. In an embodiment, the preferred length of the headband strap 3 is 26 inches.

The headband strap 3 further comprises a front plate 6 that is detachably coupled to the headband strap 3. In an embodiment T5 plastic snaps (items 9-11) are used to secure the front plate 6 onto the front of the headband strap 3. The front plate 6 is used as a cover for the front of the headband and the battery 12, which battery protrudes through a hole in the headband strap 3. The front plate 6 can be made from the same materials as the headband strap 3 but it can also be screen printed or marked with a logo or other orientation and locations markings that assist the user in placing or adjusting the headband into a position for use.

Circuit board 8 is attached to the headband strap 3 on the opposite side of the front plate 6. In an embodiment, on the forehead side of the circuit board 8, is the headband foam 7. This foam preferably is a closed-cell adhesive-backed polyethylene. This headband foam 7 includes apertures to allow light emitting diodes (LEDs) LED's and a receiver direct access to and contact with the forehead of a user. The circuit board 8 is preferably secured to the headband strap by adhesive on the headband foam 7, thus creating a strong bond between the headband strap 3 and the circuit board 8. The headband foam 7 can be sewn to the headband strap 3 for added durability.

A user may utilize the HEG headband assembly 1 by placing the headband foam 7 against the user's forehead in a left, center or right forehead location or locations. The headband may be wrapped around the user's head and attached thereto by pressing the hook and loop fabric together to secure it in place.

### Circuit Board

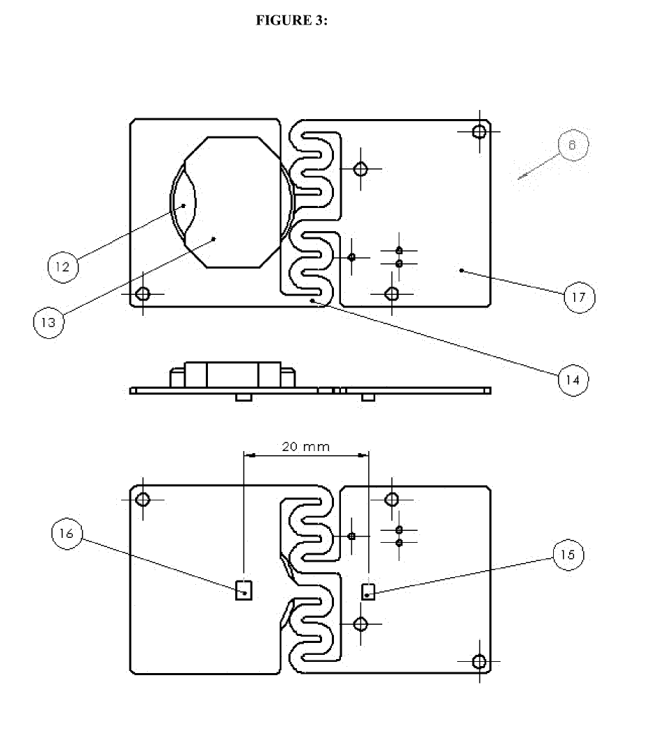

The circuit board 8 is shown in a preferred embodiment in FIG. 3. Circuit board 8 is made from PCB board 17 that is typically fiber reinforced epoxy laminated to copper sheets which are etched to carry current. Several components are mounted to the PCB board 17. Such components include a battery 12 to power the circuit. The battery is preferably a disposable CR2032 lithium coin cell. The battery 12 is mounted to the top of the PCB 17 with a battery holder 13 and protrudes through a hole or aperture in the headband strap 3.

On the bottom side of the PCB board 17 reside at least one LED pair 15 and at least one receiver 16. The at least one LED pair consists of a red LED and an infrared LED on the same chip. The red LED has a wave length of 660 nm and the infrared LED has a wavelength of 940 nm. An exemplary LED arrangement for the present disclosure is Vishay model #VSMD66694.

The at least one LED pair is capable of alternate pulsing of the red and infrared LED. The LEDs are positioned directly on the forehead so that maximum radiation can be detected by the receiver 16. The receiver 16 measures the amount of light returned from the forehead and a microprocessor calculates an activation number. The activation number represents the amount of blood flow in the trained area of the brain. In an embodiment, the measurement may be the amount of each wavelength of light reflected by cerebral blood flow in the activated cortical tissue. The data is sent to a microprocessor, which then calculates the ratio of red to infrared light and translates it into a visual signal corresponding to oxygenation level on a graphical interface the user can see.

It is very important that the circuit board 8 have some flex to it so that it conforms to varying curvatures of different foreheads and different forehead locations. This is accomplished by a serpentine flex joint 14 that incorporated into the PCB Board 17. Having a serpentine joint 14 at the center of the circuit board 8 allows the two halves of the circuit board 8 to be able to flex up to 50 degrees. This flexing is critical as it allows the at least one LED pair 15 and the receiver 16 to be more in line so that the receiver 16 can capture more of the light from the LEDs. This gives a more accurate picture of the amount of blood flow in the trained area of the brain and increases the sensitivity and accuracy of the activation number.

Another critical factor in obtaining enough signal from the at least one LED pair 15 is the spacing between the at least one LED pair 15 and the receiver 16. A spacing of 20 mm is preferred but it will be apparent that larger or smaller spacings may be used.

### Device Operation

The purpose of the device is to increase the amount of blood that can be delivered to certain parts of the brain. Blood brings fuel and oxygen to the brain and more blood flow makes for a better working brain. This disclosure primarily targets the pre-frontal cortex, which cortex resides under the forehead. This area of the brain works best with HEG neurofeedback mostly because there is no hair in the way to obstruct the light path. The pre-frontal cortex is also the area of the brain that controls attention, focus, executive planning and impulse control. This area is referred to herein as the training area.

The device allows the user to quantify the amount of blood flow in the brain at the training area. Once the user obtains this visual feedback he or she may alter the amount of blood flow to those regions of the brain. By altering this blood flow the user creates a training effect. This training effect causes the growth of new blood vessels in the training/trained areas allowing those areas of the brain to be more easily activated when called upon.

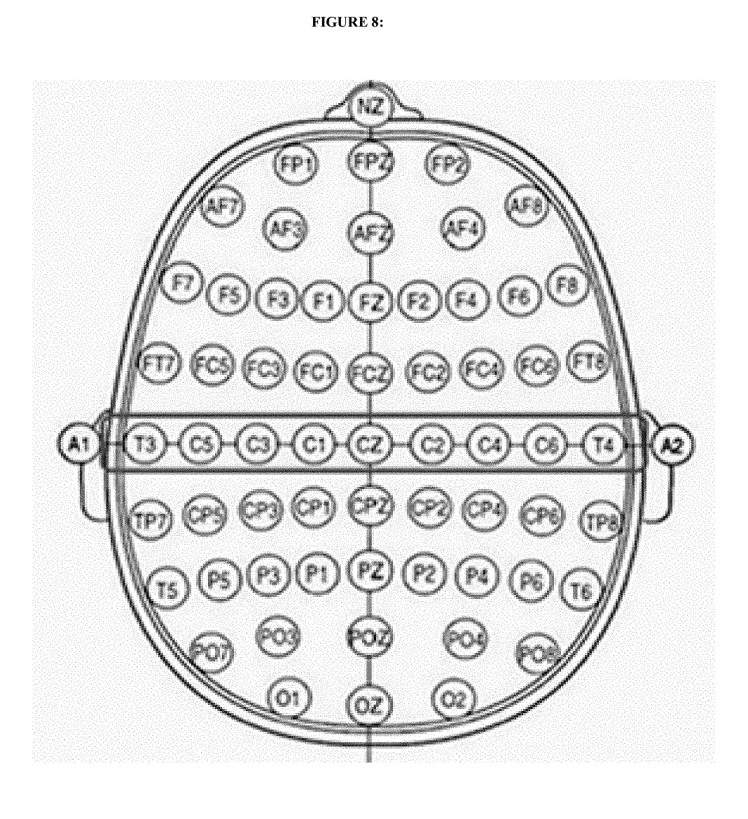

To use the device, the HEG headband assembly 1 should be placed on the forehead with the front plate 6 facing out and the headband foam placed against the forehead. In an embodiment, there are three training positions fp1, fp2 and fpz. Fp1 is on the left side of the forehead right above the eyebrow. Fp2 is the right side and Fpz is the center of the forehead. These locations can be seen in FIG. 8.

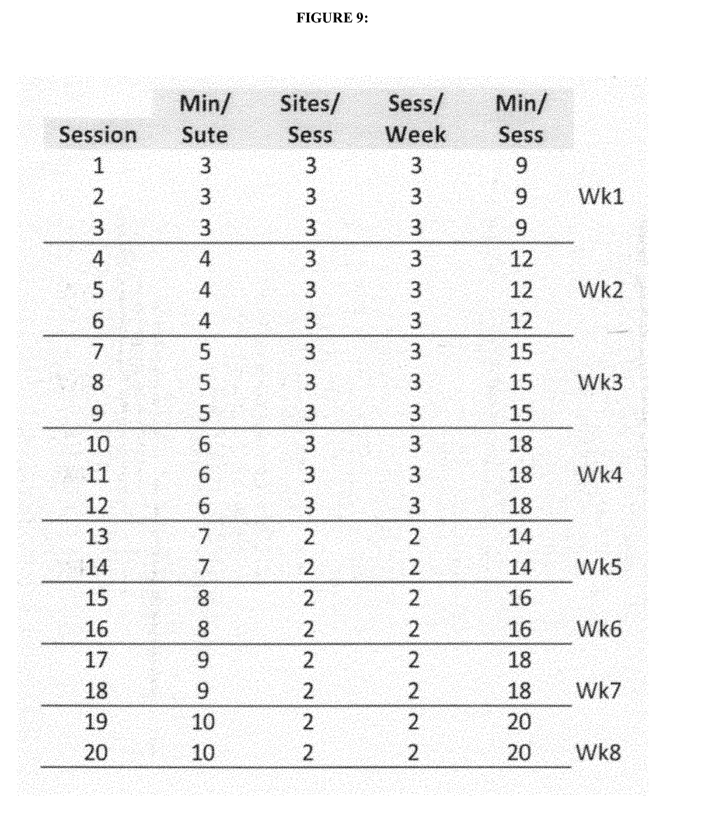

In an embodiment, two or three of the training locations (fp1, fp2 and fpz) are used during a training session. The order and locations are preferably varied from session to session to fully develop the entire pre-frontal cortex. An example training schedule is included in FIG. 9. The total training time may increase over time as the user becomes more accustom to the technology. It is preferred to space each training session at least 24 hours from one another so that the trained area can recover and grow from the training.

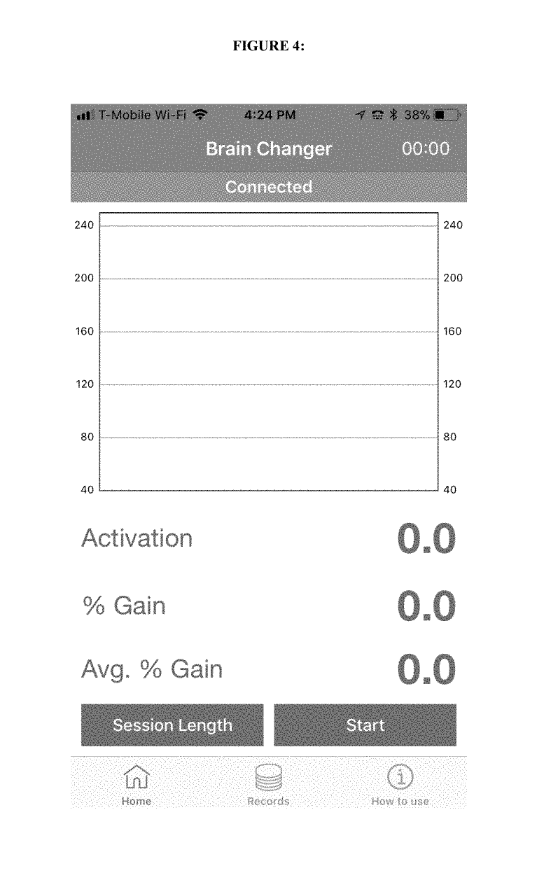

Once the HEG headband assembly 1 is secure on the forehead in either the fp1, fp2 or fpz locations the computer or mobile application commences running. Screenshots of various operational phases of the application are depicted in FIGS. 4-7. The headband assembly preferably includes a Bluetooth antenna (as part of the circuit board, for example), such that if the device (such as a computer or mobile phone or tablet) that hosts the application also has Bluetooth functionality, the device may connect the application to the headband assembly wirelessly and via Bluetooth.

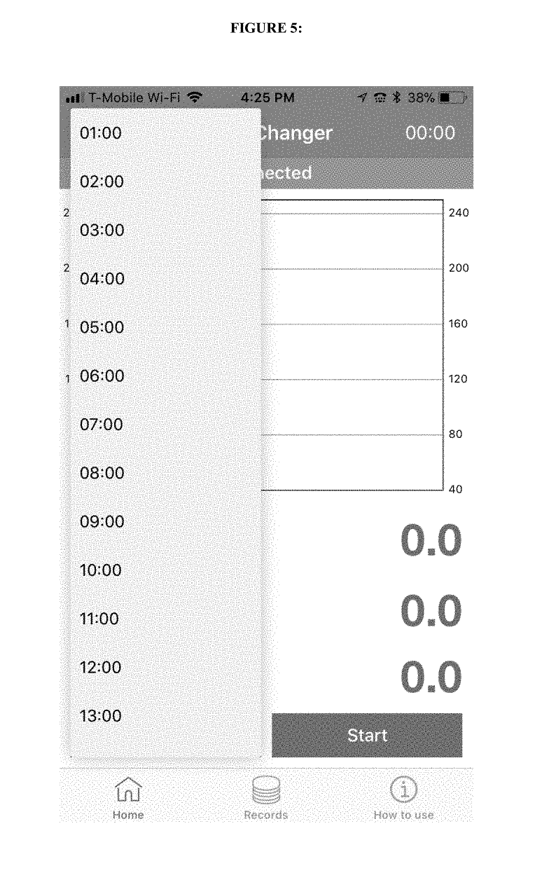

FIG. 4 shows an exemplary first screen on the application and the application connected to the HEG headband assembly 1. Once the application is so connected, the user may press the “Session Length” button as seen in FIG. 5 to select the amount of training time.

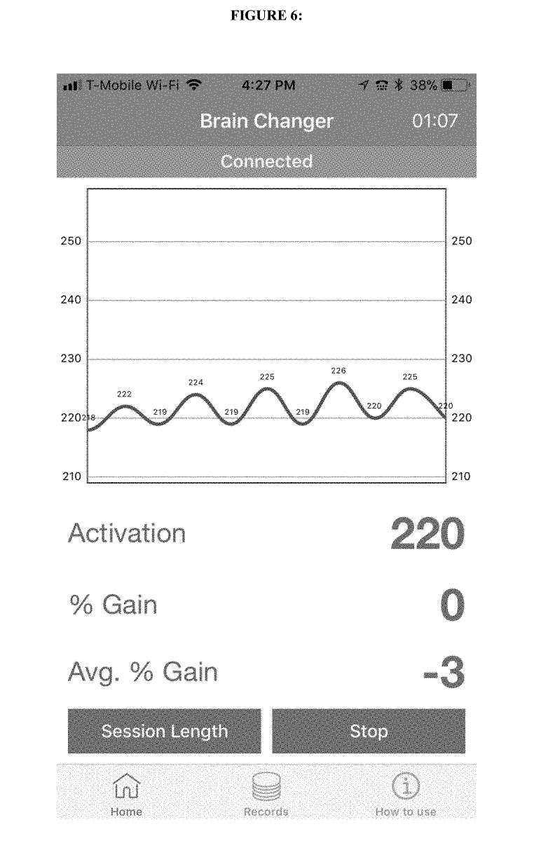

The user starts the training session by pressing the “Start” button on the application. FIG. 6 shows a graph of the data being generated by the HEG headband assembly 1.

In an embodiment, three numbers are shown on this screen. One number may refer to “activation”, which activation number may represent the amount of blood flow in the part of the brain that the headband is training. A goal of such training is to have this number increase and remain increased. A second number may refer to percentage gain, which percentage gain represents the current percentage increase in the activation number expressed as a percentage. As part of training, it is preferably for this number to increase as well. A third number is the average percentage gain. This number documents the average increase in blood flow for the entire session.

A user of the device preferably actively tries to increase all of these numbers in the interest of maximizing the productivity of a training session. Testing of the device has shown that these numbers may be increased by simply thinking about moving them up. A user may be able to accomplish this with 5-10 minutes of such thinking during his or her first training session.

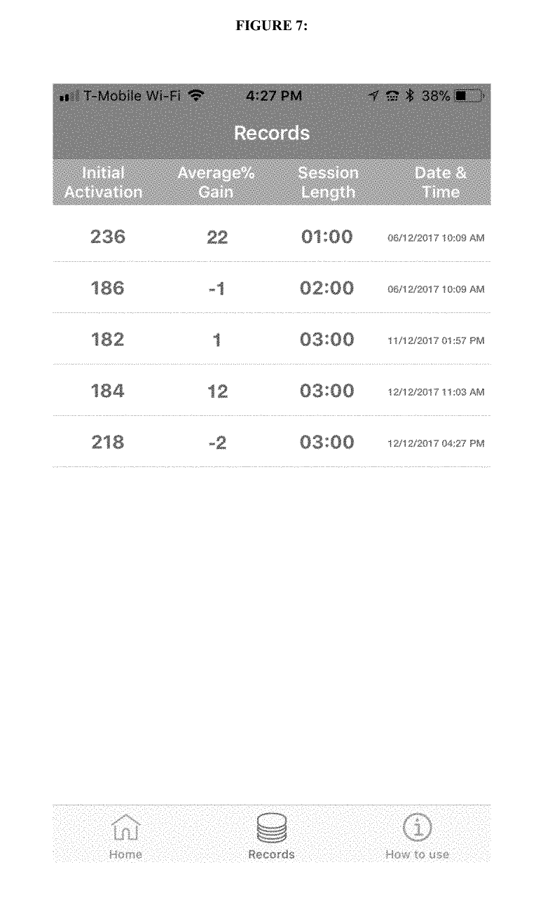

Once the “Session Length” time runs out, the data from the session is automatically recorded by the application, such as in a “user records” section of the application. The data recorded may include but is not necessarily limited to: Initial Activation, Average % Gain, Session Length and the Date and Time. Each time session may be recorded and stored on a separate line in this user records section. This can be seen in FIG. 7.

In an embodiment, the device can be used to control the user experience via video. For example, when the activation number is increasing the video can run at full playback speed. If the activation stays the same the video could reduce the playback speed notifying the user they need to increase activation. If the activation starts to decline the video could stop playing completely until the user increases blood flow and shows a rising activation number.

It is also critically important to be able to track results. It is important to know based on an objective measure how the training is working. The most accurate standard for this is the T.O.V.A. test which stands for Test of Variables of Attention. The T.O.V.A. test has been used for decades by clinicians and researchers to quantify attention. An open source version of the test called TOAV (for Test of Attentional Diligence) can also be used. Such test is 24 minutes long, broken into two halves. The test subject presses a button when a condition is presented. The condition is presented infrequently in the first half of the test and very frequently in the second half of the test. The frequency is typically at a ratio of 3 to 1.

These tests allow the quantification of response time, variability, commission (impulsive) errors and omission (inattention or distraction) errors.

Taking the test before and after a number of training sessions can help establish the amount of progress that is taking place due to the use of the HEG device.

This test and others like it could be incorporated into the app to help with these assessments.

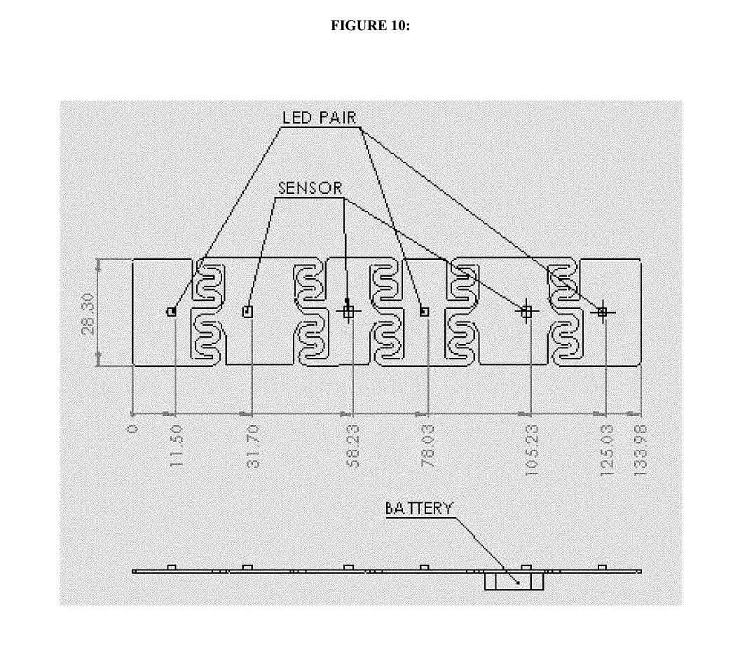

In another embodiment, and as shown in FIG. 10, an alternative HEG assembly comprises multiple pairs of LEDs and sensors (and in a further exemplary embodiment, three such pairs of LEDs and sensors) and multiple serpentine flex joints (and in a further exemplary embodiment, five such flex joints). The advantage of having multiple pairs of LEDs and sensors is that the blood flow of the entire prefrontal cortex can be measured simultaneously. This eliminates the need to move the headband location to train the entire brain.

Using multiple measurement points allows the software to narrow in on one specific location of the brain or average all of the signals to provide a more complete picture of blood flow.

The use of multiple serpentine flex joints ensures that the sensors can comfortably conform to foreheads of various sizes and contours, and accordingly, of any user.

A preferred embodiment of the device comprises 3 pairs of LEDs, 3 sensors and five serpentine flex joints (as depicted in FIG. 10.) The preferred spacing would be to evenly space the three sensors and LED pairs with the outermost sensor distance between the range of 4 to 5 inches with 4.5 inches being the preferred spacing.

Although the aforementioned elements are used in the preferred design, it is understood by those familiar with the art that considerable simplification is possible without departing from the spirit of the disclosure. Further, although there have been described particular embodiments of the present disclosure it is not intended that such references be construed as limitations upon the scope of this disclosure. The preferred embodiments of the disclosure described here are exemplary and numerous modifications, variations and rearrangements can be readily envisioned to achieve an equivalent result, all of which are intended to be embraced within the scope of the appended claims.

What is claimed is:

1. A hemoencephalography feedback device, the device comprising
a headband assembly, said headband assembly comprising a headband strap, and a circuit board attachably coupled to said headband strap,
said circuit board comprising a battery, at least one pair of light emitting diodes, at least one receiver, and a microprocessor,
said at least one pair of light emitting diodes comprising a red light and an infrared light,
said at least one receiver capable of recording measurements and
said microprocessor capable of calculating measurements taken by the receiver,
wherein, when the pair of light emitting diodes are positioned directly on a user's forehead, the light emitting diodes emit light upon the user's forehead and the receiver measures the amount of light returned from the forehead and a microprocessor calculates an activation number that represents the amount of blood flow in the area of the user's forehead.

2. The device of claim 1, wherein said circuit board is flexible.

3. The circuit board of claim 2, wherein said circuit board comprises a serpentine flex joint allowing the circuit board to be flexed at least 15 degrees.

4. The circuit board of claim 2, wherein the circuit is sewn into the headband assembly.

5. The device of claim 1 further comprising a software application, and said circuit board of claim 1 further comprising wireless means of coupling with said software application.

6. The device of claim 1, wherein the device comprises multiple pairs of light emitting diodes and receivers, said multiple pairs of light emitting diodes and receivers being disposed at regular intervals along the headband assembly.

7. The device of claim 1, wherein the device comprises multiple pairs of light emitting diodes and receivers, said multiple pairs of light emitting diodes and receivers being disposed at selected physiological intervals along the headband assembly.

8. The device of claim 1, wherein the device controls the function of a video.

9. A system for measuring hemoencephalograpy feedback, the system comprising
a headband assembly, said headband assembly comprising a headband strap, and a circuit board attachably coupled to said headband strap,
said circuit board comprising a battery, at least one pair of light emitting diodes, at least one receiver, and a microprocessor,
said at least one pair of light emitting diodes comprising a red light and an infrared light,
said receiver capable of recording measurements and
said microprocessor capable of calculating measurements taken by the receiver,
a display device operatively coupled to the microprocessor.
wherein, when the pair of light emitting diodes are positioned directly on a user's forehead, the light emitting diodes emit light upon the user's forehead, the receiver measures the amount of light returned from the forehead, a microprocessor calculates an activation number that represents the amount of blood flow in the area of the user's forehead, and the display device displays the calculation of the microprocessor.

10. The device of claim 9, wherein said circuit board is flexible.

11. The circuit board of claim 10, wherein said circuit board comprises a serpentine flex joint allowing the circuit board to be flexed at least 15 degrees.

12. The device of claim 9, wherein the device comprises multiple pairs of light emitting diodes and receivers, said multiple pairs of light emitting diodes and receivers being disposed at regular intervals along the headband assembly.

13. The device of claim 9, wherein the device comprises multiple pairs of light emitting diodes and receivers, said multiple pairs of light emitting diodes and receivers being disposed at selected physiological intervals along the headband assembly.

14. The display device and microprocessor of claim 8, wherein the coupling of said display device and microprocessor is via wireless means.

15. A hemoencephalograpy feedback device, the device comprising
a headband assembly, said headband assembly comprising a headband strap, and a circuit board attachably coupled to said headband strap,
said circuit board comprising a battery, at least one pair of light emitting diodes, at least one receiver, and a microprocessor,
said circuit board comprises at least one serpentine flex joint,
said receiver capable of recording measurements and
said microprocessor capable of calculating measurements taken by the receiver,
wherein, when the pair of light emitting diodes are positioned directly on a user's forehead, the light emitting diodes emit light upon the user's forehead and the receiver measures the amount of light returned from the forehead and a microprocessor calculates an activation number that represents the amount of blood flow in the area of the user's forehead.

16. The device of claim 15, wherein the device comprises multiple pairs of light emitting diodes and receivers, said multiple pairs of light emitting diodes and receivers being disposed at selected intervals along the headband assembly.

17. The device of claim 16, wherein the device comprises three pair of light emitting diodes and three receivers, said multiple pairs of light emitting diodes and receivers being disposed at selected intervals along the headband assembly with the outermost sensors spaced between 4 and 5 inches apart.
